(19) United States
(12) Patent Application Publication
(10) Pub. No.: US 2014/0097847 A1
(43) Pub. Date: Apr. 10, 2014

(54) OPTICAL ANGULAR MOMENTUM INDUCED HYPERPOLARISATION IN INTERVENTIONAL APPLICATIONS

(71) Applicants: Daniel Robert Elgort, New York, NY (US); Lucian Remus Albu, Forest Hills, NY (US)

(72) Inventors: Daniel Robert Elgort, New York, NY (US); Lucian Remus Albu, Forest Hills, NY (US)

(73) Assignee: KONINKLIJKE PHILIPS N.V., EINDHOVEN (NL)

(21) Appl. No.: 14/123,656

(22) PCT Filed: Jun. 11, 2012
(86) PCT No.: PCT/IB2012/052935

§ 371 Date: Dec. 5, 2013

Related U.S. Application Data
(60) Provisional application No. 61/497,110, filed on Jun. 15, 2011.

(51) Int. Cl. A61B 5/055; G01R 33/46

(52) U.S. Cl. CPC A61B 5/055; G01R 33/46
USPC 324/322

(57) ABSTRACT

A magnetic resonance spectroscopy assembly includes a magnet to generate a steady magnetic field, an RF transmit/receive antenna to transmit an RF excitation field into an examination region and acquire magnetic resonance signals from the examination region and a magnetic resonance spectrometer coupled to the RF transmit/receive antenna to collect magnetic resonance spectroscopy data from the magnetic resonance signals. An interventional instrument is provided with the assembly. The interventional instruments carries an optical module to generate photonic radiation endowed with orbital optical momentum (OAM).

### FIELD OF THE INVENTION

The invention pertains to a magnetic resonance spectroscopy assembly including a magnet to generate a steady magnetic field and a magnetic resonance spectrometer to collect magnetic resonance spectroscopy data.

### BACKGROUND OF THE INVENTION

Such a magnetic resonance assembly is known from the paper The use of 1-H magnetic resonance spectroscopy in inflammatory bowel diseases: distinguishing ulcerative colitis from Crohn's disease. Bezabeh T, Somorjai R L, Smith I C, Nikulin A E, Dolenko B, Bernstein C N. 2001, Am J Gastroenterol, Vol. 96, pp. 442-448.

The known magnetic resonance assembly uses proton(¹H) magnetic resonance spectroscopy to detect early inflammation of the gastrointestinal tract of tissue samples of small animals. In particular, the known magnetic resonance assembly is able to differentiate between Crohn's disease and ulcerative colitis.

### SUMMARY OF THE INVENTION

An object of the present invention is to provide a magnetic resonance assembly that allows access to the small intestines to acquire magnetic resonance signals. This object is achieved by the magnetic resonance assembly including
a magnet to generate a steady magnetic field
an RF transmit/receive antenna to transmit an RF excitation field into an examination region and acquire magnetic resonance signals from the examination region
a magnetic resonance spectrometer coupled to the RF transmit/receive antenna to collect magnetic resonance spectroscopy data from the magnetic resonance signals and
an interventional instrument carrying
an optical module to generate photonic radiation endowed with orbital optical momentum (OAM).

The photonic radiation endowed with orbital angular momentum couples with molecules and atoms in tissue that is irradiated with the OAM photonic radiation. As a consequence, nuclear magnetic hyperpolarisation is generated in the irradiated tissue. From these hyperpolarised nuclei, magnetic resonance signals can be generated by applying an RF excitation field by the RF T/R antenna and subsequently receiving magnetic resonance signals with the RF T/R antenna. The magnet generates a stationary magnetic field to establish a nuclear processional frequency. Typically, the field strength of the stationary magnetic field is in the range of 0.05-3 T.

These and other aspects of the invention will be further elaborated with reference to the embodiments defined in the dependent Claims.

The optical module to generate the OAM light can be built small enough to fit in the distal end (catheter tip) of an interventional instrument. This is achieved in that a photonic, e.g. optical, source beam is brought to the tip of the device via a fibre optic waveguide. A set of miniature optical elements are arranged at the tip of the fibre, which include: polarisers, beam expander (to enable the beam to fill a forked hologram), a diffractive grating with the forked hologram pattern, a spatial filter (to select the diffraction component with the OAM), and focusing lenses. To ensure the optical system works for high values of the optical angular momentum of the photonic beam (1-values, the size of the spatial filter and the aperture of the other optical elements will need to be increased in accordance with the radius of the photonic beam with OAM increasing with 1-value). As a relatively weak stationary magnetic field is needed only to establish the precession frequency of the hyperpolarised nuclei (i.e. hyperpolarised nuclear spin moments), only a simple magnet is sufficient which can be employed outside of the body of the patient to be examined or may even be integrated in the distal end of the interventional instrument. From the acquired magnetic resonance signals magnetic resonance spectral data are derived by the magnetic resonance spectrometer. In this way the invention enables to access the small intestines to perform magnetic resonance spectroscopy locally to gather data which enable a physician to assess the state of health in the small intestines. The generation of the magnetic resonance signals from the OAM photonic beam is known per se from the international application WO 2009/081360-A1.

In an aspect of the invention, the optical module combines the functions of generating OAM photonic radiation to generate hyperpolarisation of the tissue, with optical imaging of that tissue. The optical imaging can also be employed to navigate the interventional instrument through the anatomy, such as the gastrointestinal tract, of the patient to be examined.

In another aspect of the invention, a rotatable or moveable reflector, e.g. a rotatable of movable mirror or prism is employed to switch the optical module between optical imaging and generating OAM photonic radiation. The purpose of the rotatable prisms, or mirrors could be used instead, are so that the photonic beam can be sent out the distal end of the interventional instrument with OAM or without OAM (without OAM it will presumable be used for illuminating the anatomy in front of the interventional instrument to aid visual inspection or video imaging). Preferably, several prisms can be employed, where one of the prisms may have its position physically translated or rotated so that it no longer blocks the photonic beam coming out of the fibre optic wave guide.

In a further embodiment of the invention, the RF T/R antenna is formed by a micro coil that is mounted on the distal end of the interventional instrument. Such a small sized micro coil can be mounted on the distal end of the interventional instrument which is thin enough to be able to navigate through the small intestines. , For example the micro-coil’ size may be in the range of 4-20 mm diameter, An arrangement of multiple (e.g. three orthogonal) MR coils would be advantageous to ensure that the interventional instrument has sensitivity to the MR signal, which resides in the plane perpendicular to the static magnetic field. In clinical practice, the physical orientation of the endoscope relative to the static field may change during the procedure, so a set of three orthogonal coils will endure that the full MR signal can be reconstruct. Alternatively, the set of coils could be a two orthogonal loop coils, possibly with multiple turns to increase the inductance of the coil, to provide sensitivity to the left/right and to the top/bottom of the tip at the distal end of the interventional instrument, and a solenoid coil to provide sensitivity in front of the tip. In an alternative embodiment of the invention, the RF T/R antenna is formed by an surface coil that can be placed on the patient's body, in close proximity to the region to be examined, and thus close to the position of the distal end of the interventional instrument. Thus, the interventional instrument does not need to carry the RF T/R micro coil and can be smaller so that is navigates through the small intestines easier.

These and other aspects of the invention will be elucidated with reference to the embodiments described hereinafter and with reference to the accompanying drawing wherein

### BRIEF DESCRIPTION OF THE DRAWINGS

FIG. 1 shows a schematic representation of the magnetic resonance spectroscopy assembly of the invention and

FIG. 2 shows a schematic representation of details of the optical module of the magnetic resonance assembly of the invention.

### DETAILED DESCRIPTION OF THE EMBODIMENTS

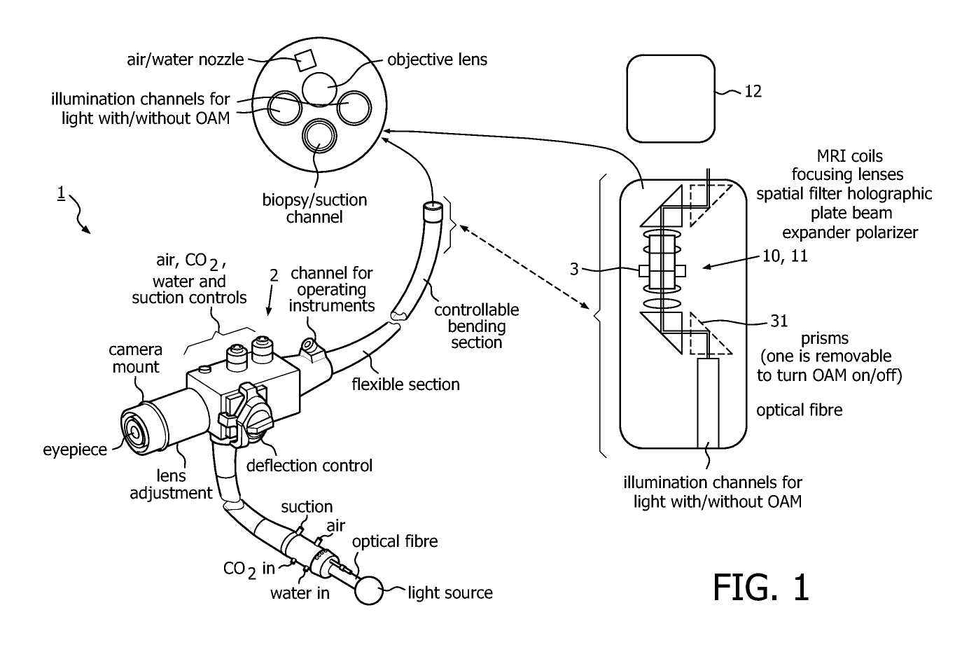

FIG. 1 shows a schematic representation of the magnetic resonance spectroscopy assembly of the invention. In this example the magnetic resonance spectroscopy assembly 1 is integrated in part in the interventional instrument 2. At the distal end of the interventional instrument 2, i.e. the part that is inserted in the body of the patient to be examined, the optical module 3 is mounted with the magnet 10 to generate a steady magnetic field and RF transmit/receive antenna 11 to acquire the magnetic resonance signals generated by the OAM photonic beam. A magnetic resonance spectrometer 12 is coupled to the output of the RF transmit receive antenna. The magnetic resonance spectrometer 12 incorporates a digital signal acquisition system (DAS) and a magnetic resonance spectrometer 12. The DAS receives the signals acquired by the RF coil and converts them into digital signals that are input to the magnetic resonance spectrometer 12 which derives magnetic resonance spectral data from the input digital signals. On the basis of the magnetic resonance spectral data a magnetic resonance spectrum can be displayed. Because the signals acquired by the RF coil originate from hyperpolarised tissue generated by the OAM photonic beam produced by the optical module, the magnetic resonance spectrum represents the compounds in the hyperpolarised tissue. Thus, the magnetic resonance spectrometer 12, incorporated (in part) in the interventional instrument is able to generate a local magnetic resonance spectrum of the tissue at the distal end of the interventional instrument. Thus, the invention achieves to acquire a magnetic resonance spectrum from the internal anatomy of a patient in a minimal invasive manner. In the example shown, the distal end is formed as a controllable bending section that can easily navigate through the patient's anatomy.

A light source is provided at the proximal end of the interventional instrument and optical fibres are provided to guide the light from the light source to the optical module 3.

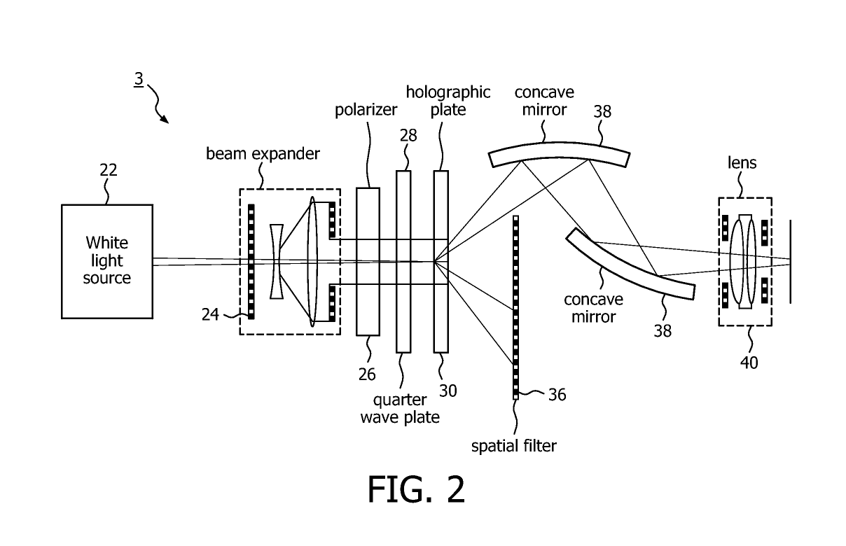

FIG. 2 shows a schematic representation of details of the optical module of the magnetic resonance assembly of the invention. With reference now to FIG. 2, an exemplary arrangement of optical elements is shown for endowing light with OAM. It is to be understood that any electromagnetic radiation can be endowed with OAM, not necessarily only visible light. The described embodiment uses visible light, which interacts with the molecules of interest, and has no damaging effect on living tissue. Light/radiation above or below the visible spectrum, however, is also contemplated. A white light source 22 produces visible white light that is sent to a beam expander 24. In alternate embodiments, the frequency and coherence of the light source can be used to manipulate the signal if chosen carefully, but such precision is not essential. The beam expander includes an entrance collimator 251 for collimating the emitted light into a narrow beam, a concave or dispersing lens 252, a refocusing lens 253, and an exit collimator 254 through which the least dispersed frequencies of light are emitted. In one embodiment, the exit collimator 254 narrows the beam to a 1 mm beam.

After the beam expander 24, the light beam is circularly polarized by a linear polarizer 26 followed by a quarter wave plate 28. The linear polarizer 26 takes unpolarised light and gives it a single linear polarization. The quarter wave plate 28 shifts the phase of the linearly polarized light by ¼ wavelength, circularly polarizing it. Using circularly polarized light is not essential, but it has the added advantage of polarizing electrons.

Next, the circularly polarized light is passed through a phase hologram 30. The phase hologram 30 imparts OAM and spin to an incident beam. The value “1” of the

OAM is a parameter dependent on the phase hologram 30. In one embodiment, an OAM value 1=40 is imparted to the incident light, although higher values of 1 are theoretically possible. The phase hologram 30 is a computer generated element and is physically embodied in a spatial light modulator, such as a liquid crystal on silicon (LCoS) panel, 1280×720 pixels, 20×20 μm2, with a 1 μm cell gap. Alternately, the phase hologram 30 could be embodied in other optics, such as combinations of cylindrical lenses or wave plates. The spatial light modulator has the added advantage of being changeable, even during a scan, with a simple command to the LCoS panel.

Not all of the light that passes through the holographic plate 30 is imparted with OAM and spin. Generally, when electromagnetic waves with the same phase pass through an aperture, it is diffracted and projected into a pattern of concentric circles some distance away from the aperture (Airy pattern). The bright spot (Airy disk) in the middle represents the 0th order diffraction, in this case, that is light with no OAM. Circles adjacent the bright spot represent diffracted beams of different harmonics that carry OAM. This distribution results because the probability of OAM interaction with molecules falls to zero at points far from the centre of the light beam or in the centre of the light beam. The greatest chance for interaction occurs on a radius corresponding to the maximum field distribution, that is, for circles close to the Airy disk. Therefore, the maximum probability of OAM interaction is obtained with a light beam with a radius as close as possible to the Airy disk radius.

With reference to FIG. 2, a spatial filter 36 is placed after the holographic plate to selectively pass only light with OAM and spin. An example of such a filter is shown in FIG. 5. The 0th order spot 32 always appears in a predictable spot, and thus can be blocked. As shown, the filter 36 allows light with OAM to pass. Note that the filter 36 also blocks the circles that occur below and to the right of the bright spot 32. Since OAM of the system is conserved, this light has OAM that is equal and opposite to the OAM of the light that the filter 36 allows to pass. It would be counterproductive to let all of the light pass, because the net OAM transferred to the target molecule would be zero. Thus, the filter 36 only allows light having OAM of one polarity to pass.

With continuing reference to FIG. 2, the diffracted beams carrying OAM are collected using concave mirrors 38 and focused to the region of interest with a fast microscope objective lens 40. The mirrors 38 may not be necessary if coherent light were being used. A faster lens (having a high f-number) is desirable to satisfy the condition of a beam waist as close as possible to the size of the Airy disk. In alternate embodiments, the lens 40 may be replaced or supplemented with an alternative light guide or fibre optics.

1. A magnetic resonance spectroscopy assembly including
a magnet to generate a steady magnetic field
an RF transmit/receive antenna to transmit an RF excitation field into an examination region and acquire magnetic resonance signals from the examination region
a magnetic resonance spectrometer coupled to the RF transmit/receive antenna to collect magnetic resonance spectroscopy data from the magnetic resonance signals and
an interventional instrument carrying an optical module to generate photonic radiation endowed with orbital optical momentum (OAM).

2. A magnetic resonance spectroscopy assembly as claimed in claim 1, wherein the optical module combines the functions of (i) generation of photonic radiation endowed with orbital momentum and (ii) optical imaging of an field of view around the interventional instrument's distal end.

3. A magnetic resonance spectroscopy assembly as claimed in claim 2, wherein the optical module includes a rotatable reflector, in particular a rotatable prism between an OAM-orientation and an imaging orientation, the optical module generating OAM endowed photonic radiation with the prism in its OAM-orientation and the optical module imaging its field of view.

4. A magnetic resonance spectroscopy assembly as claimed in claim 1, wherein the magnet is integrated in the interventional instrument.

5. A magnetic resonance spectroscopy assembly as claimed in claim 1, wherein a RF receive/transmit coil is integrated in the interventional instrument and the RF receive/transmit coil is coupled to the magnetic resonance spectrometer.

6. A magnetic resonance spectroscopy assembly as claimed in claim 1, comprising a surface RF receive/transmit coil or coil array which is coupled to the magnetic resonance spectrometer.
